(19) United States
(12) Patent Application Publication
(10) Pub. No.: US 2024/0091417 A1
(43) Pub. Date: Mar. 21, 2024

(54) ANTI-STAINING HYDRATION MEDIUM AND MEDICAL PRODUCTS CONTAINING THE SAME

(71) Applicant: Hollister Incorporated, Libertyville, IL (US)

(72) Inventors: Satwinder S. Panesar, Foxford (IE); David J. Farrell, Ballina (IE)

(21) Appl. No.: 17/768,033

(22) PCT Filed: Oct. 13, 2020
(86) PCT No.: PCT/US2020/055406

§ 371 Date: Apr. 11, 2022

Related U.S. Application Data
(60) Provisional application No. 62/914,731, filed on Oct. 14, 2019.

(51) Int. Cl. A61L 29/14; A61L 29/02

(52) U.S. Cl. CPC A61L 29/14; A61L 29/02; A61L 2400/10

(57) ABSTRACT

Anti-staining hydration mediums for medical products and packaged medical products containing the same.

The present application claims the benefit of and priority to U.S. Provisional Patent Application No. 62/914,731, filed on Oct. 14, 2020, which is hereby incorporated herein by reference.

### DESCRIPTION

### Technical Field

The present disclosure generally relates to hydration mediums for hydrating medical devices, and more particularly to, anti-staining hydration mediums that reduce or do not stain fabric. The present disclosure also relates to medical products containing such hydration mediums.

### Background

Some medical devices are required to be hydrated with a hydration medium or stored in a hydration medium, such as liquid water or saline. Such devices may include hydrophilic urinary catheters, contact lenses, etc. These medical devices may be packaged in a package containing the medical device and the hydration medium. When the package is opened, there is a risk that the hydration medium unintentionally spills or leaks from the package. If the hydration medium contacts the user's clothing, the hydration medium may leave a stain on the fabric of the clothing.

Intermittent hydrophilic urinary catheters include a hydrophilic coating on the outer surface of the catheter. When the hydrophilic coating is wetted or hydrated with a hydration medium, such as water, it becomes extremely lubricous which eases introduction of the catheter into the body and aids in reducing pain and discomfort associated with such introduction.

Intermittent catheter users typically perform self-catheterization about six times a day. Intermittent hydrophilic catheters may be provided in a package containing the catheter and a hydration medium. Several intermittent catheter users have limited dexterity, which can make it difficult for such users to manipulate and open the package. When the catheter package is opened, the hydration medium may inadvertently leak or spill from the package, increasing the risk that the hydration medium may come into contact with the user's clothing and stains the fabric of the clothing.

Self-catheterization is a very personal procedure, but due to the activities of the user, self-catheterization is oftentimes performed outside of the home. For example, active users may need to self-catheterize in a public bathroom. In these situations, users tend to prefer to be discreet about self-catheterizing. When the user's clothing becomes stained from hydration fluid, it reduces the privacy and discreetness of the procedure. Because of this, several catheter packages include structural features that attempt to retain the hydration medium within the package. However, these structural features do not guarantee that the hydration medium will not leak/spill out of the package.

Therefore, there remains a need for packaged medical devices that include a hydration medium and reduces the risk of the hydration medium staining the user's clothes.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a hydration medium for hydrating medical devices that includes monomeric polyol(s) in an amount of between about 1 wt % and about 10 wt %, a surfactant in an amount between about 0.01 wt % and about 10 wt % and hydration liquid being the balance of the hydration medium. The hydration medium having a viscosity that is at most about 10 cP (or at most about 5 cP), and a surface tension of the hydration medium that is at most about 65 mN/m.

In another aspect, a packaged medical product includes a medical product and a hydration medium. The hydration medium includes monomeric polyol(s) in an amount of between about 1 wt % and about 10 wt %, a surfactant in an amount between about 0.01 wt % and about 10 wt % and hydration liquid being the balance of the hydration medium. The hydration medium having a viscosity that is at most about 10 cP (or at most about 5 cP), and a surface tension of the hydration medium that is at most about 65 mN/m.

In another aspect, a hydration medium for hydrating medical devices includes a solution having a viscosity that is at most about 10 cP (or at most about 5 cP), and a surface tension that is at most about 65 mN/m.

### BRIEF DESCRIPTION OF FIGURES

FIG. 1 is a plan view of a catheter assembly in accordance with the present disclosure.

### DETAILED DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

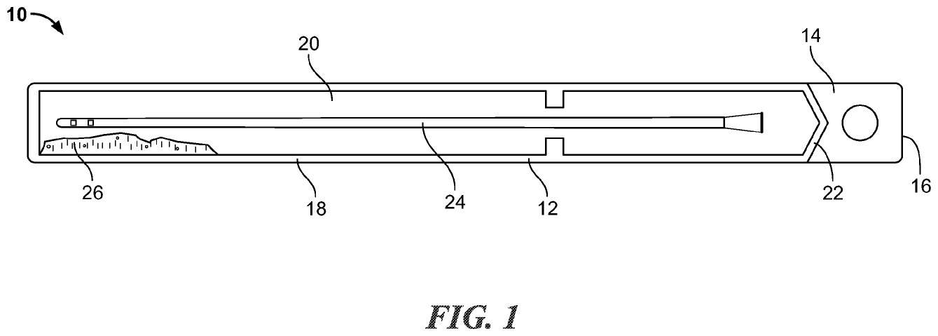

The present disclosure discloses anti-staining hydration mediums, such as hydration liquids, that reduce staining or do not stain fabric of clothing. The present disclosure also discloses medical device products including a package containing a medical device and a hydration medium. One exemplary medical device product 10 is shown in FIG. 1. In this FIGURE, the medical device is illustrated as a urinary catheter 24. However, the medical device may be any other suitable medical device that is packaged with a hydration medium, such as liquid water or saline. The packaged hydrophilic medical device product 10 includes a package 12 that surrounds that catheter 24. In the embodiment illustrated in FIG. 1, the package 12 is formed from a front sheet 14 and back sheet 16 that are sealed together to form a peripheral seal 18 and define an internal cavity 20. At the top of the package 12, the front and back sheets 14 and 16 may be unattached above top seal 22. The package 12 may be opened by grasping these unattached portions and pulling the front sheet 14 and back sheet 16 away from each other to peal open the package 12 along seal 18. Optionally, the package 12 may be any other suitable package for containing a medical device. For example, the package may be a tear open package. Additionally, the material of the package, optionally, may be made from a gas impermeable material.

A hydrophilic medical device is contained within cavity 20. As mentioned above, in the illustrated embodiment, the hydrophilic medical device is a hydrophilic catheter 24. The hydrophilic catheter 24 may be any suitable hydrophilic catheter that includes a hydrophilic outer surface that becomes lubricous when hydrated with a hydration medium, such as water. For example, the catheter 24 may include a lubricious hydrophilic coating on the outer surface of the catheter.

A hydration medium, such as a hydration liquid 26, also may be located within the cavity 20. The hydration medium may be loose or free flowing within the cavity 20, or the hydration medium may be in a compartment within the cavity 20 wherein the compartment keeps the hydration medium stored separately from the catheter 24. During manufacturing or just prior to use, the separate compartment may be opened so that the hydration medium 26 is free flowing with the cavity 20 and contacts the catheter.

The hydration medium may be an anti-staining hydration liquid 26, which reduces staining or does not stain fabrics with which the hydration liquid comes into contact. In one embodiment, for example, the hydration medium may be a solution of liquid water and other components. The solution may have a viscosity which allows the hydration medium to seep and/or penetrate through fabrics (e.g., cotton, linen, polyester or denim fabrics). In one embodiment, the solution has a viscosity that is at most about 10 cP. In an alternative, the viscosity may be between about 1 cP and about 9 cP, between about 5 cP and bout 10 cP, between about 5 cP and about 8 cP, or at most about 5 cP. The viscosity may be measured by Ubbelohde or Anton Paar SVM 3001 viscometers, primarily kinematic viscosity and multiplied by density to obtain dynamic viscosity.

In addition to having a viscosity that assists in allowing hydration liquid to seep and/or penetrate through fabrics, or in alternative to this, the hydration liquid may have a surface tension that allows the hydration medium to seep and/or penetrate through fabrics. In one embodiment, the surface tension is at most about 65 mN/m. In one embodiment, the surface tension may be at most about 45 mN/m. In one alternative, the surface tension of the hydration liquid may be between about 45 mN/m and about 65 mN/m. The surface tension may be measured by tensiometer or goniometer (such as those made by Kruss gmbh).

In one embodiment, the hydration liquid may have any combination of viscosity and surface tension discussed above. For example, in one alternative, the viscosity is at most 10 cP and the surface tension is at most 64 mN/m. In another alternative, the viscosity may be between about 1 cP and about 9 cp and the surface tension may between about 45 mN/m and about 64 mN/m.

The hydration medium may include a solution of water and one or more components that may be used to tune the viscosity and/or surface tension. For example, the hydration medium may include a component to tune the viscosity of the solution. In one alternative, the solution includes one or more monomeric polyol(s) in an amount that is at most about 10 wt %. In an alternative, the amount of monomeric polyol(s) may be between about 1 wt % and about 10 wt %. In one embodiment, the monomeric polyol(s) may be at most about 10 wt % and the viscosity may at most 10 cP. In another embodiment, the monomeric polyol(s) may be between about 1 wt % and about 7 wt % and the viscosity may be between about 1 cp and about 9 cP. The monomeric polyols may be one or more of glycerol, erythritol, ethylene glycol, diethylene glycol, propylene glycol, 1-4 butanediol and sucrose.

In an alternative or in addition to the viscosity tuning component, the solution may also include a surface tension tuning component. For example, the solution may include a surfactant in an amount that is at most 10 wt % of the solution. In one alternative, the surfactant may be in an amount between about 2 wt % and about 10 wt %. In one embodiment, the surfactant may be at most about 10 wt % and the surface tension may be at most 65 mN/m. In another embodiment, the surfactant may be between about 0.01 wt % and about 10 wt % and the surface tension may be between about 45 mN/m and about 65 mN/m. The surfactant may be one or more of an anionic, non-ionic, cationic and amphoteric surfactant.

In one embodiment of the hydration medium, the hydration medium includes combination of the above described viscosity tuning agents and viscosities and surfactants and surface tensions. For example the hydration medium may include monomeric polyol(s) in an amount of between about 0.01 wt % and about 10 wt % and a surfactant in an amount between about 2 wt % and about 10 wt %, with hydration liquid or hydration liquid with other additives being the balance of the hydration medium. Also, the hydration medium has a viscosity that is at most about 10 cP, and a surface that is at most about 65 mN/m.

The other additives may be for example, osmolality increasing agents, gelling agents, antimicrobials, preservative agents, polymers, polysaccharides, gums (xanthan, carrageena, agar, arabic, xanthan etc.), mannose, sugar, trehalose polyethylene glycol, Sodium chloride, urea, hydrogen peroxide, citric acid, hypochlorous acid etc.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A hydration medium for hydrating medical devices, comprising:
a monomeric polyol(s) in an amount of between about 1 wt % and about 10 wt %;
a surfactant(s) in an amount between about 0.01 wt % and about 10 wt %;
hydration liquid being the balance of the hydration medium; and
wherein a viscosity of the hydration medium is at most about 10 cP, and a surface tension of the hydration medium is at most about 65 mN/m.

2. The hydration medium of claim 1, wherein the viscosity of the hydration medium is between about 1 cP and about 9 cP.

3. The hydration medium of claim 1, wherein surface tension of the hydration medium is at most about 45 mN/m.

4. The hydration medium of claim 1, wherein the surface tension of the hydration medium is between about 45 mN/m and about 65 mN/m.

5. The hydration medium of claim 1, wherein the monomeric polyol(s) comprise one or more of glycerol, erythritol, ethylene glycol, diethylene glycol, propylene glycol, 1-4 butanediol and sucrose.

6. The hydration medium of claim 1, wherein the surfactant(s) comprises one or more of an anionic, non-ionic, cationic and amphoteric surfactant.

7. The hydration medium of claim 1, wherein the hydration medium comprises water.

8. The hydration medium of claim 1, wherein the hydration medium comprises saline.

9. The hydration medium of claim 1, further comprising additional agents.

10. A packaged medical product, comprising:
a medical product;
a hydration medium comprising:
a monomeric polyol(s) in an amount of between about 1 wt % and about 10 wt %;
a surfactant(s) in an amount between about 0.01 wt % and about 10 wt %;
hydration liquid being the balance of the hydration medium; and
wherein a viscosity of the hydration medium is at most about 10 cP, and a surface tension of the hydration medium is at most about 65 mN/m.

11. The packaged medical product of claim 10, wherein the medical product comprises a hydrophilic intermittent urinary catheter.

12. The packaged medical product of claim 10, wherein the viscosity of the hydration medium is between about 1 cP and about 9 cP.

13. The packaged medical product of claim 10, wherein surface tension of the hydration medium is at most about 45 mN/m.

14. The packaged medical product of claim 10, wherein the surface tension of the hydration medium is between about 45 mN/m and about 65 mN/m.

15. The packaged medical product of claim 10, wherein the monomeric polyol(s) comprise one or more of glycerol, erythritol, ethylene glycol, diethylene glycol, propylene glycol, 1-4 butanediol and sucrose.

16. The packaged medical product of claim 10, wherein the surfactant(s) comprises one or more of an anionic, non-ionic, cationic and amphoteric surfactant.

17. The packaged medical product of claim 10, wherein the hydration medium comprises water.

18. The packaged medical product of claim 10, wherein the hydration medium comprises saline.

19. The packaged medical product of claim 10, further comprising additional agents.
